(19) United States
(12) Patent Application Publication
(10) Pub. No.: US 2024/0019239 A1
(43) Pub. Date: Jan. 18, 2024

(54) SENSOR STRIP AND DEVICE FOR MEASURING GEOMETRIC SHAPES

(71) Applicant: Minktec GmbH, Braunschweig (DE)

(72) Inventors: Benjamin Holmer, Braunschweig (DE); Martin Schwerter, Braunschweig (DE); Eugen Koch, Braunschweig (DE); Tom Schroder, Braunschweig (DE)

(73) Assignee: Minktec GmbH, Braunschweig (DE)

(21) Appl. No.: 18/032,914

(22) PCT Filed: Oct. 29, 2021
(86) PCT No.: PCT/EP2021/000137

§ 371 Date: Apr. 20, 2023

(30) Foreign Application Priority Data
Oct. 29, 2020 (DE) 10 2020 006 662.3

(51) Int. Cl. G01B 7/16; G01L 1/20; G01L 1/22

(52) U.S. Cl. CPC G01B 7/20; G01L 1/205; G01L 1/2287

(57) ABSTRACT

The present invention relates to a flexible sensor strip for measuring geometric shapes, in particular bending radii or the like, and to an associated device which can process and evaluate sensor signals of the sensor strip. The sensor strip comprises a substrate, on which a plurality of resistor pairs is arranged. Each of these resistor pairs has a resistor on the substrate front side and a further resistor on the substrate rear side. Both of these resistors are connected in series and between the poles of a supply voltage so that they form a voltage divider. The special feature of the present invention is that an electrical via and/or a pair of electrically interconnected contact elements is provided for the series circuit, that is, for the connection between the two resistors. When the substrate or the sensitive region of the sensor strip is moved into itself, in particular in the event of bending and/or twisting (torsion), the mid voltages in the affected voltage dividers change. This is sensed and evaluated by the measuring device according to the invention.

The present invention relates to a flexible sensor strip for measuring geometric shapes, such as, in particular, bending radii or the like. It is hereby also possible by means of a suitable evaluating apparatus to measure and to evaluate the associated time course of these geometric shapes and thus to determine corresponding movements, such as, in particular, bendings, twistings (torsions), and/or stretchings.

Sensor strips of the kind mentioned are fundamentally known and often contain a plurality of electrical elements, which can function as resistors and/or capacitors and which are placed on a flexible substrate. When such a sensor strip experiences suitable mechanical deformations, such as bendings, twistings (torsions), stretchings, compressions, or the like, individual ones of these electrical elements can be stretched and others compressed. Changes in the resistance or capacitance values ensuing from this can be detected by means of an evaluation unit. Suitable algorithms can be used to determine where and how one of the mentioned mechanical loads arises.

Moreover, the utilization of sensor strips of this kind on human and animal bodies is known. Thus, the unexamined published German patent application (Offenlegungsschrift) DE 10 2008 052 406 A1 relates to a method for recording functional parameters for characterizing the course of movements on human or animal bodies as well as a bending sensor for carrying out the method. In an embodiment therein, strain gauges are provided, which serve for recording stretching deformations in that, even for minor deformations, they change their impedance, such as, in particular, their electrical resistance. There hereby exists the problem, however, that tensile or compressive stresses that arise under certain circumstances cause changes in length, which are interpreted falsely as a measure for the bending of the object of investigation. In order to prevent this, a tension-resilient, yet elastically bendable substrate is preferably provided therein, such as, for example, spring steel. Used for an electrical insulation of such a substrate from the strain gauges that are put in place on the substrate is a layer of adhesive, such as for example, epoxy resin.

The published international patent specification WO 2011/032575 A1 relates to a method and a system for recording functional parameters for characterizing the course of movements on the human body, such as, in particular, in the region of the lumbar spine, as well as a method for analyzing such functional parameters. To this end, bending sensors are used, in which strain gauges are fastened on a substrate, that is, for example, adhesively fastened on a substrate. Proposed as substrate materials in this case are, among other things, an electronic circuit board or a strip of spring steel. The realization therein of the electrical contact between the strain gauges and the strip conductors on the circuit board occurs, for example, by soldering a strip of copper foil.

In an exemplary embodiment therein, the bending sensor comprises a plurality of strain gauges, two of which are fastened to opposite sides of the substrate in each case in such a way that the strain gauges record the same bending of the substrate that traces the bending or flexion of the object of examination, such as, for example, a human back.

It is also described therein that, in this way, it is possible to compensate for interference values by use of a bridge circuit, such as, for example, a Wheatstone bridge, which forms a differential signal from the signals of the two strain gauges, and to amplify the actual measurement signal. In this way, it is possible to compensate for the influence of errors, such as tensile or compressive stresses, as well as of temperature fluctuations, which, under certain circumstances, can cause additional changes in the length of the substrate.

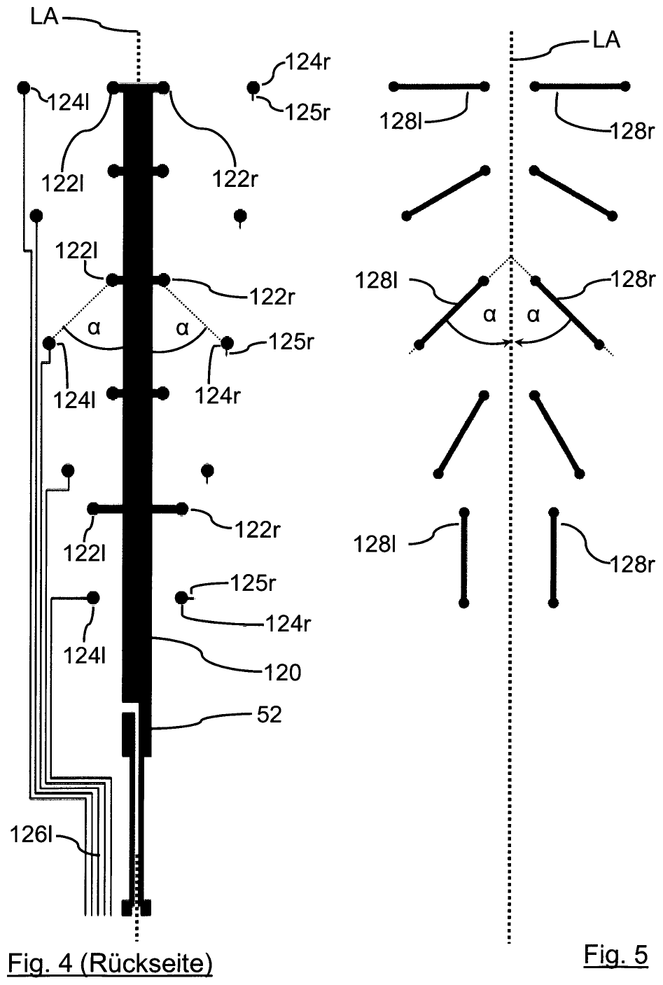

The published international patent specification WO 2016/030752 A1 relates to a stretchable and flexible sensor as well as an associated system for measuring and processing data relating to movements, such as, for example, those of a back or limb of a person or an animal. A plurality of strips (3) are hereby fastened to an elastic film strip (2), such as, for example, by stitching, adhesive attachment, clamping, etc. Also proposed therein is a special design (FIG. 4), in which two groups of parallel strips form a fishbone pattern. These strips can be designed as capacitors or as elastic single wires having a length-dependent resistance.

It ensues from the cited documents that hitherto known sensor strips of the kind mentioned are tedious to realize and are also prone to error.

The problem of the present invention is therefore to present a sensor strip that can be produced in a simple and low-cost manner and has a long lifetime.

The sensor strip according to the invention is defined by the main claim. Advantageous further developments are defined by the dependent claims. The subsequent device claims relate to an associated measuring and evaluating device.

The sensor strip according to the invention comprises a flexible substrate, which, in particular, can be bent and/or twisted (subjected to torsion). A film made of polyethylene terephthalate (PET) has proven especially expedient for this purpose, the thickness of which can lie in the range of 10 to 1000 μm and preferably in the range of 100 to 400 μm and most preferably at about 300 μm. Of course, other flexible materials are also conceivable, such as, for example, polyurethanes (PU), thermoplastic polyurethane (TPU), polyethylene terephthalate (PET), polyamide (PA), other plastics, and/or other suitable materials.

Present on the substrate is at least one first resistor pair. Provided on one of the substrate sides, which is referred below as the front side, is a first electrical resistor. Provided on the other substrate side, which is referred to below as the rear side, is a second resistor. These two resistors are designed and arranged in such a way that they lie essentially opposite to each other. However, slight deviations relating to the geometry and/or position are hereby possible and are also to be expected, in particular, on account of manufacturing tolerances. It has been found that deviations in position up to certain values are well possible and still afford rather good results. Corresponding tolerance values are dependent on, among other things, the thickness of the substrate and/or the value of the resistance. It has been found that deviations in position up to approximately 1 to 2 mm and/or up to approximately 10% of the resistance value are tolerable. It is hereby also possible in the scope of the subsequent signal evaluation to make corresponding electronic corrections.

The first terminal of the first resistor is electrically connected to a first supply voltage conductor, via which it is connected to the first pole of a supply voltage. The first terminal of the second resistor is electrically connected to a second supply voltage conductor, via which it is connected to the second pole of the supply voltage. This second pole can be electrically connected to the ground terminal of an electrical unit to which the sensor strip is connected.

The special feature of the sensor strip according to the invention is that the second terminals of the resistors are interconnected electrically, so that the resistors form a voltage divider. This can be realized, for example, in that at least one via is provided for the electrical connection between the second terminals of the two resistors. In the intendment of the present invention, such a via is any kind of electrical connection that extends through an opening in the substrate between the front side and the rear side and, moreover, electrically connects a first strip conductor on the substrate front side to a second strip conductor on the substrate rear side. Furthermore, the first strip conductor is hereby in electrical contact with the second terminal of the first resistor and the second strip conductor is hereby in electrical contact with the second terminal of the second resistor. These electrical strip conductors can be identical or different and can also be designed in any manner.

An electrical connection between the second terminals of the resistors can also occur in accordance with the invention in that the first strip conductor on the substrate front side and/or the second strip conductor on the substrate rear side extend or extends into an edge region of the substrate and is or are designed and arranged there in such a way that each of them has a contact site, via which they can be electrically connected. Such an electrical terminal is preferably realized in that, in normal operation—that is, when the sensor strip is ready to carry out measurements—a first contact element is in electrical contact with the first strip conductor and, in normal operation, a second contact element is in electrical contact with the second strip conductor. The two contact elements can be part of a plug connection, a clamp connection, a dual contact ZIF connector (ZIF=zero insertion force), a solder connection, or the like. The mentioned contact elements are electrically interconnected, with it being possible for this to occur within the associated plug connector or else outside of it, such as, for example, in a connected conductor or in a following control device.

Such a sensor strip according to the invention is very compact and is also easy to produce. In standby normal operation, the mentioned supply voltage is applied, so that a voltage divider is realized, with the mid voltage being applied to the second terminals of the resistors. The value thereof depends, on the one hand, on the supply voltage and, on the other hand, on the ratio of the resistance values (first resistance, second resistance) to each other. Preferably, the two resistance values are of equal magnitude when the substrate is situated in a plane without any mechanical load, such as, for example, in the plane of the drawing. When the substrate in itself is now moved, such as, for example, by a bending and/or a twisting (torsion), the resistance values change. This change is dependent on various factors, such as the geometric shape of the resistor, the kind of movement, and the position of the resistors in relation to the location of movement.

Although, in the following description of the present invention, it is assumed that the base surfaces of the resistors—that is, the respective surface in relation to the substrate—have a length that is substantially greater than their width, the invention is in no way limited to resistors of this kind. When such an oblong resistor is now stretched such that its length is increased, its resistance value becomes larger. In the case of a compression with an associated decrease in the length, its resistance value becomes smaller. In the case of the described sensor strip according to the invention, this effect can be used with only one resistor pair, for example, in order to measure an arching, a diameter, or the like in a body. Such a body can be an object, such as, for example, a rod, a ball, or the like. However, it is also possible to measure parts of a human body or of an animal body. It is also possible to include the bending of body parts, such as the bending of a back, a knee, or the like. Such a measurement can occur one time. However, it is also possible for a plurality of such measurements to be carried out in succession, as a result of which a dynamic course can be determined This can also comprise, in particular, the courses of speed and/or acceleration of geometric changes. To this end, the mid voltages are recorded by means of a suitable evaluation unit at predetermined intervals in time and/or at predetermined times and associated measured values are formed and evaluated.

In a further development of the sensor strip according to the invention, at least one second resistor pair is provided. In this case, too, a first resistor is arranged on the substrate front side and a second resistor is arranged on the substrate rear side. The first terminal of the first resistor is electrically connected to the first pole of the supply voltage and the first terminal of the second resistor is electrically connected to the second pole of the supply voltage. The second terminals of the two resistors are electrically interconnected, so that a voltage divider is formed. However, it is hereby possible, but not necessarily provided, that, for the electrical connection between the second resistor terminals, at least one via or at least one electrical contacting of the above-mentioned kind is present. This second resistor pair is very similar to the first resistor pair, so that, in addition, reference is also made to the above description.

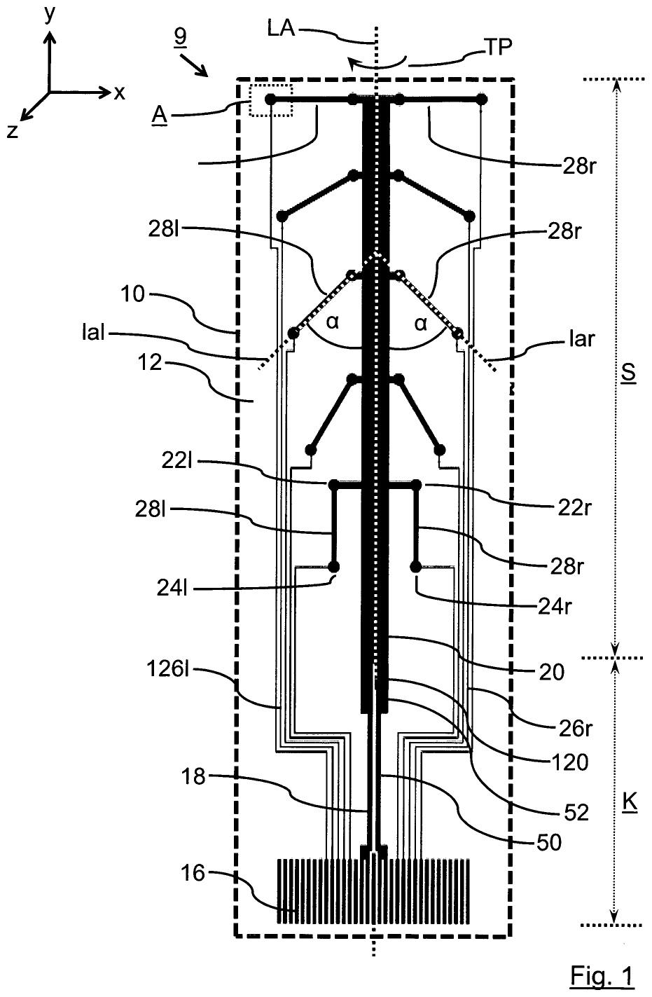

It ensues from the mentioned embodiments that different resistor pairs are possible, namely, one or a plurality of the first resistor pairs and one or a plurality of the second resistor pairs. These are specially arranged with respect to one another. This will be described below. To this end, a Cartesian coordinate system is used for specifying positions and directions, such as shown, for example, in FIG. 1 and from which the following ensues:
the x-y plane corresponds to the plane of the drawing
the x axis is directed from left to right
the y axis is directed from bottom to top
the z axis points outward out of the plane of the drawing.

It is assumed below that the substrate is arranged in such a way that it extends along the x-y plane; that is, it lies virtually in the plane of the drawing. Such a state is also referred to below as the resting state. It is then possible to distinguish the following cases for the present invention.
a) A first resistor pair and a second resistor pair

When only one of the first resistor pairs and only one of the second resistor pairs are present, then they are arranged adjacent to each other. This means that the first resistor pair, for example, lies to the left of the second resistor pair (x direction). The two resistor pairs can hereby lie at the same height or at different height (y direction).
b) A plurality of the first resistor pairs and/or a plurality of the second resistor pairs

When two or a plurality of the first resistor pairs are present, they are arranged one above the other (y direction). It is hereby possible that that are laterally offset (x direction) with respect to each other. When two or a plurality of the first* resistor pairs are present, they are likewise arranged one above the other (y direction) and can also be laterally offset (x direction).

When two or a plurality of both the first resistor pairs and the second resistor pairs are present in each case, the first resistor pairs are situated adjacent to (for example, to the left of) the second resistor pairs, with an offset of adjacently lying resistor pairs in the y direction being possible.
c) Different number of resistor pairs

For completeness, it is mentioned that the number of first resistor pairs can be different from the number of second resistor pairs.

For the further geometric description, it is assumed that a normal that extends between the adjacently lying resistor pairs and along the y axis can be defined. This normal can correspond, for example, to the longitudinal axis of the sensor strip, such as is provided further below in connection with the description of preferred exemplary embodiments.

Preferably, at least individual ones of the mentioned resistors have an oblong base surface. This means that they have a length that is markedly greater than their width. It is possible to determine from this an associated resistor longitudinal axis, which is used below to define an angle of inclination between the resistor pairs and the mentioned normals. This angle can have a value between zero and 90 degrees (inclusive in each case), with values between 20 and 40 degrees and, in particular, a value of approximately 30 degrees having proven especially expedient. It is hereby possible for the values of this angle of inclination to be the same in size for all resistor pairs. However, it is also possible for the values to be different.

The value of the mentioned angle of inclination determines, in particular, the kind of possible measurement with the associated resistor pair. This will be addressed in detail further below in the description of the exemplary embodiments.

When, for a specific number of first resistor pairs, an equal number of second resistor pairs is arranged in mirror symmetry with respect to the normals, the sensor strip is especially well suited to measure torsions.

In order to be able to produce a sensor strip according to the invention in a low-cost and nonetheless reliable manner, it has proven expedient to produce the resistors—or at least individual ones thereof—by a screen printing method. To this end, a high-ohmic paste is applied to the substrate, such as, for example, a carbon-based paste, a CNT-containing paste, an electrically conductive polymer (for example, PEDOT), or the like. In the case of carbon screen printing, a thickness in the range of approximately 5 to 20 μm is preferably used. In the case of other materials, it is also possible to use other thicknesses.

Furthermore, it is possible to produce strip conductors—or at least individual ones thereof—and/or at least individual ones of the mentioned vias by a screen printing method using a low-ohmic paste, such as, for example, a silver paste, a copper paste, or the like.

In order to be able to produce the sensor strip according to the invention as effectively and at as lowest cost as possible, it has proven expedient to use a single resistor layer (carbon layer) and/or a single conductive layer (silver layer). These layers are nearly symmetrical around the mentioned y axis (longitudinal axis) and can be used both for the printing on the substrate front side and for the printing on the substrate rear side.

The sensor strip according to the invention can be subdivided into a sensor region, in which the mentioned resistors are arranged, and a contact region, in which electrical terminals for an evaluation unit and/or for the above-mentioned electrical contact connections between the first strip conductors on the substrate front side and the second strip conductors on the substrate rear side can be produced, such as, for example, by means of a plug connection, a clamp connection, or a solder connection and/or by electrical adhesive. Usually, in normal operation—that is, when an object (or body) is to be measured—the contact region is less deformed than the sensor region. This can occur in various ways, such as, for example, by way of the design of the substrate, by way of the present electrical connection to the evaluation unit (in particular, by a plug connector), and/or by way of corresponding fastening of the sensor strips to the object that is to be examined. As a result of this, the contact region is also subject to less mechanical load than the sensor region. Therefore, it is especially advantageous to arrange at least individual ones of the vias in this contact region in order to increase the reliability.

As already mentioned, the sensor strip according to the invention is provided in order to be part of a measuring device that can measure and evaluate the geometric shapes and/or the dynamic movements of objects, human bodies, animal bodies, etc. To this end, the sensor strip is connected to an evaluation unit via suitable electrical connections. These connections deliver the supply voltage and receive, as sensor signals, the mid voltages produced by the resistor pairs functioning as voltage dividers, evaluate them, and then produce an output signal, which is a measure for the changes in resistance within the individual resistor pairs and accordingly also a measure for deformations and/or movements that the sensor strip experiences.

Because such changes in resistance and accordingly also the associated measured changes in voltage are quite small, the evaluation unit can also generate a reference voltage, the value of which essentially corresponds to the voltage that the voltage divider delivers in the resting state in each case. From the reference voltage and the voltages measured in each case, a differential voltage is formed, which is amplified and evaluated.

In a further preferred embodiment, the evaluation unit contains a time-controlled switch (multiplexer), which feeds the mid voltages delivered by the individual voltage dividers in succession to further processing stages, such as amplification stages, analog/digital converters, etc. The evaluation unit also comprises memory storage components, in which are deposited values that are a measure for the position or change in position of the individual resistor pairs within the sensor strip. From the individual mid voltages and the associated positional data, an output signal is produced, which is a measure for the geometric shape and graphical reproduction of the sensor strip.

In a further preferred embodiment, the evaluation unit also comprises one time stage or a plurality of time stages, which actuates or actuate the multiplex switch and/or the further processing stages at predetermined times and/or after predetermined intervals in time in such a way that the mid voltages delivered by the individual voltage dividers are processed. Dynamic movements or courses of movement of the object that is to be examined (body) can thereby be created, such as, in particular, courses, speeds, and/or accelerations of movements. The output signal produced by the evaluation unit can accordingly contain this information as well.

In a further preferred embodiment, the evaluation unit also comprises a transmission stage. This transmission stage is fed a signal that is a measure for the produced output signals. A high-frequency signal is preferably produced from said signal, such as, for example, a Bluetooth or a WLAN signal, which can be received and further processed by a suitable device, such as a tablet computer, a smartphone, a PC, or the like. It is hereby possible for an evaluation, a memory storage, and/or a display of the output signal to occur via a suitable algorithm (such as an app or the like).

Further details and advantages of the present invention are explained below on the basis of exemplary embodiments with associated figures. Hereby shown are:

FIG. 1 a first sensor strip

FIG. 2 front-side strip conductors of the first sensor strip [Vorderseite32 Front side]'

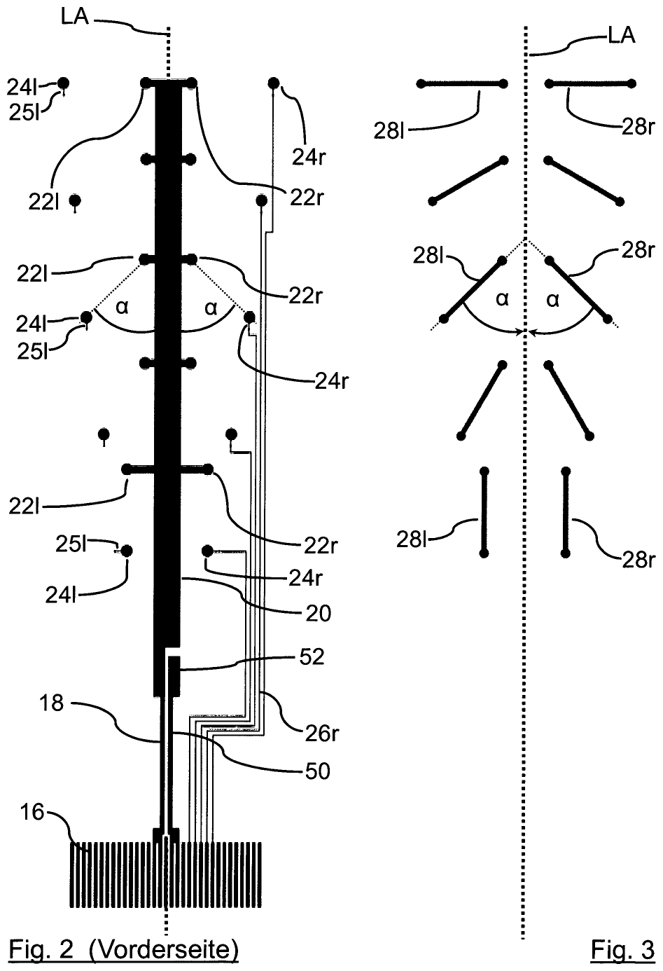

FIG. 3 front-side sensor resistors of the first sensor strip

FIG. 4 rear-side strip conductors of the first sensor strip [Rückseite=Rear side]*

FIG. 5 rear-side sensor resistors of the first sensor strip

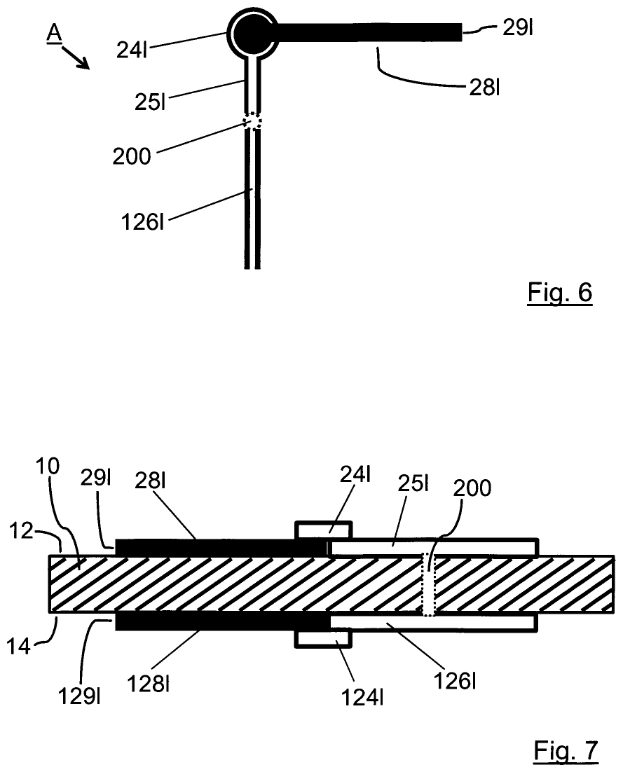

FIG. 6 symbolic plan view of a via between a front-side strip conductor and a rear-side strip conductor

FIG. 7 symbolic cross-sectional illustration of the via between a front-side strip conductor and a rear-side strip conductor

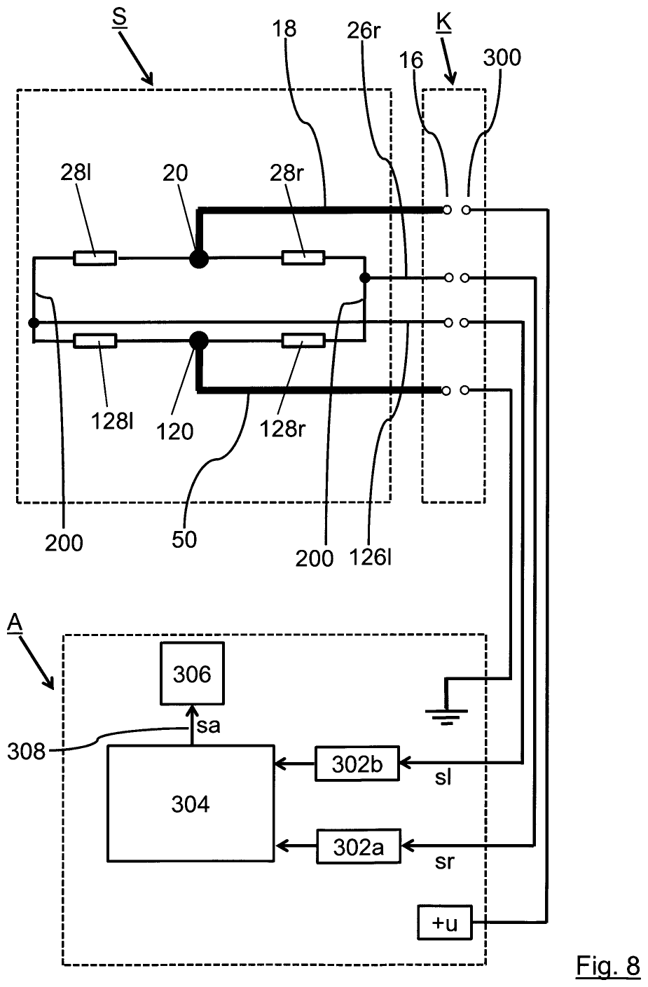

FIG. 8 circuit diagram

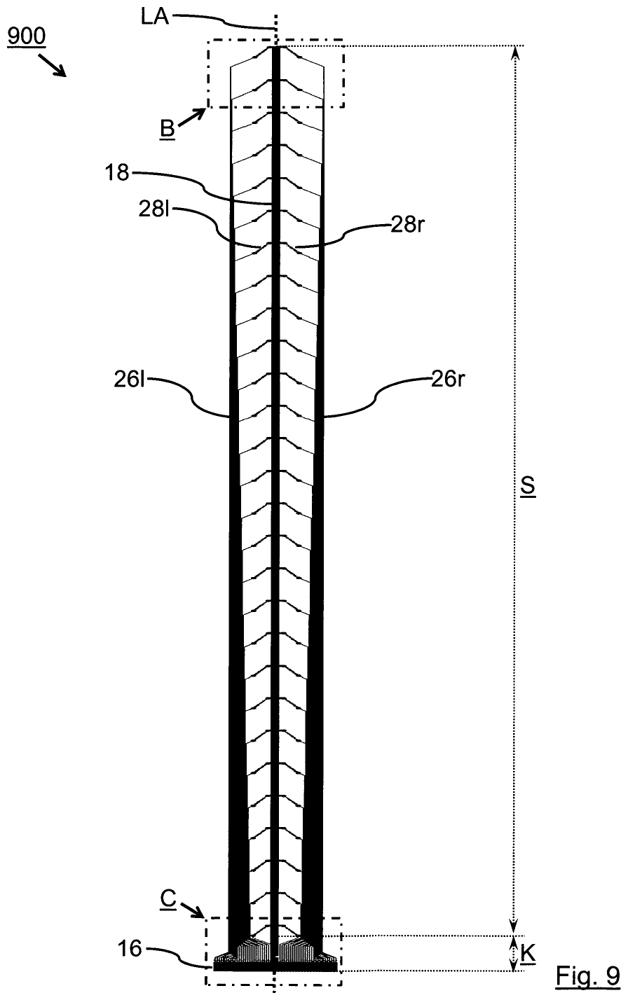

FIG. 9 a second sensor strip

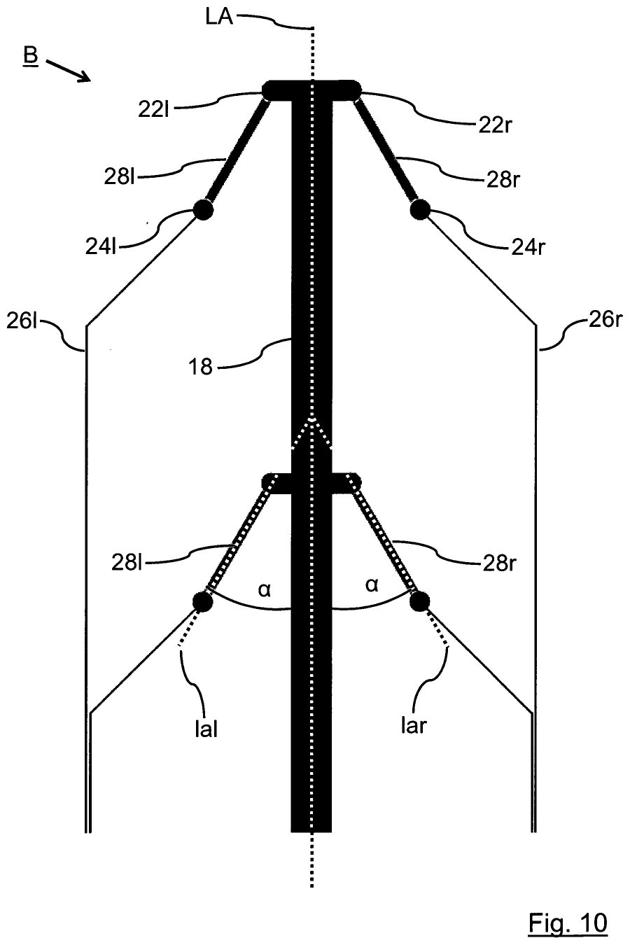

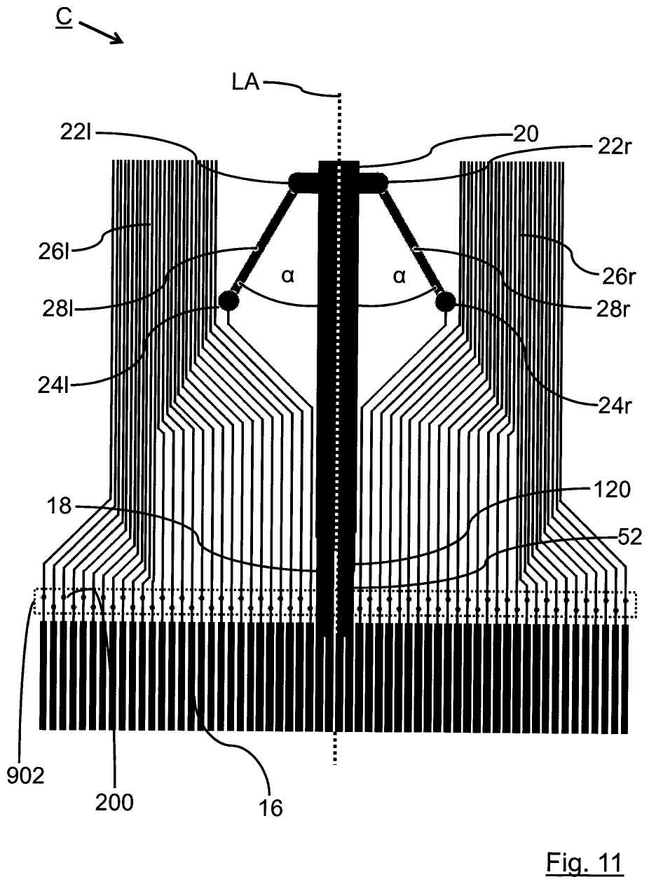

FIG. 10 the upper section B of the second sensor strip

FIG. 11 the lower section C of the second sensor strip

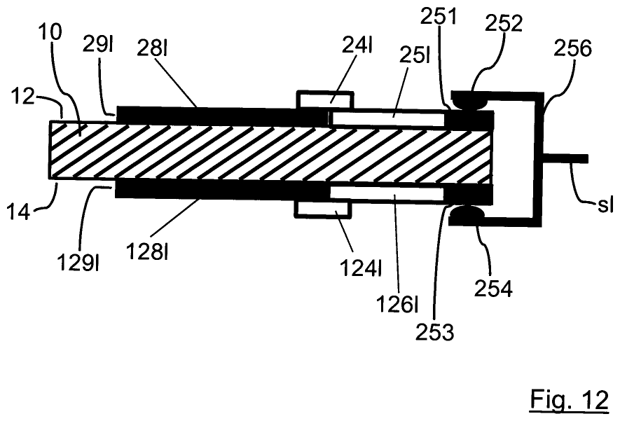

FIG. 12 symbolic cross-sectional illustration of the connection between a front-side strip conductor and a rear-side strip conductor through contact elements that are interconnected

FIG. 1 shows a Cartesian coordinate system (top left), a plan view of a first sensor strip 9 and, on the right side, a symbolic marking for distinguishing a sensor region S as well as a contact region K of the sensor strip 9. The latter is situated here within the x-y plane and accordingly in the plane of the drawing. It contains a substrate 10, which consists of a material that is bendable, twistable, and/or stretchable as well as, in the exemplary embodiment shown here, transparent. A PET film (PET=polyethylene terephthalate), the thickness of which, in a preferred embodiment, lies in the range of 100 to 180 μm, has proven to be especially expedient. The substrate 10 has a front side 12 (see FIGS. 1 and 7) as well as a rear side 14 (see FIG. 7). The front side 12 could also be referred to as the top side. However, this term is avoided here, because the positional specifications “top,” “bottom,” “right,” and “left” (and the like) serve for the description of position within the plane of the drawing. Arranged both on the front side 12 and on the rear side 14 are a plurality of sensor resistors and also a plurality of electrical strip conductors. They are described in detail below on the basis of FIGS. 1 to 5.

FIG. 2 shows those strip conductors that are arranged on the front side 12 of the substrate 10 and FIG. 3 shows the sensor resistors of the front side 12. Situated in the lower region are a plurality of electrical contact sites 16, which form a connector power strip and are designed and arranged in such a way that they can be contacted with membrane connectors (not depicted here). Two of these contact sites 16 are connected via a strip conductor 18 to a front-side supply voltage conductor strip 20, which extends here essentially along the longitudinal axis LA.

Via associated connections, contact points 22r to the right of the supply voltage conductor strip 20 and contact points 22l to the left thereof are electrically connected to the supply voltage conductor strip 20.

Belonging to each of the contact points 22r is an associated contact point 24r, which is electrically connected via an associated strip conductor 26r to a respective one of the contact sites 16. Depicted in FIGS. 1 and 2 are five of the contact point pairs 22r, 24r, only two of which are provided with reference signs (see FIG. 2). Extending between each of these contact point pairs 22r, 24r is one of five sensor resistors 28r, which here have an oblong rectangular shape.

In the exemplary embodiment shown, the uppermost of these sensor resistors 28r hereby extends in such a way that it and accordingly also its longitudinal axis form a right angle with the supply voltage conductor strip 20 and therefore also with the longitudinal axis LA. The lowermost of these sensor resistors 28r extends essentially parallel to the supply voltage conductor strip 20 and accordingly also to the longitudinal axis LA. Drawn in the case of the middle one of the sensor resistors 28r in FIG. 1 is its longitudinal axis lar and also an angle of inclination α, which is defined by the two longitudinal axes LA and lar and is approximately 45 degrees.

Situated to the left of the upper supply voltage conductor strip 20 and accordingly of the longitudinal axis LA is, besides the already mentioned contact points 22l, also associated contact points 24l, each of which is connected to a strip conductor piece 25l. Each of these strip conductor pieces 25l is connected by way of a special via to a strip conductor 126l, which is situated on the substrate rear side 14 and to the left of the longitudinal axis LA. Details regarding this are described further below.

Similarly to the right side, there are here also five contact point pairs 22l 24l, between which associated sensor resistors 28l are arranged. In this exemplary embodiment, the left sensor resistors 28l are arranged in mirror symmetry with respect to the right sensor resistors, so that the above-mentioned explanations apply analogously also to the left side.

FIG. 4 shows the strip conductors that are arranged on the rear side of the substrate 10 and FIG. 5 shows the associated sensor resistors. The direction of view on the elements shown in FIGS. 4 and 5 is from the top, that is, from the front side 12 passing through the substrate 10, with the elements that are on the substrate front side 12 not being present. Therefore, one could also say that here one sees the rear sides of those elements that are placed on the substrate rear side 14. Most of these rear-side elements cannot be seen in FIG. 1, because they are covered up by elements of the front side. Exceptions are the already mentioned strip conductors 1261.

As can be seen in FIG. 4, the substrate rear side 14 has a lower voltage supply conductor strip 120. It is connected via a strip conductor 50 (see FIGS. 1 and 2), which extends on the substrate front side 12, as well as by way of a via in the region 52, which is not depicted here, to two of the contact sites 16, which, in this exemplary embodiment, are all situated on the front side 12. The design and arrangement of the rear-side supply conductor strip 120 above the region 52 is essentially identical to the design and arrangement of the front-side supply voltage conductor strip 20. This also means, in particular, that, on the rear side 14, a plurality of contact points 122r, 1221 are present, which correspond to the contact points 22r, 221— apart from the fact that they are connected to different supply voltage conductor strips. Furthermore, five contact points 1241 are present to the left of the rear-side supply voltage conductor strip 120 and are each connected via a respective one of the left strip conductors 1261 and, in addition, by way of a via, which is not shown here, between the front side 12 and the rear side 14, to one of the front-side contact sites 16 (see FIG. 1). Situated to the right of the supply voltage conductor strip 120 are a five further contact points 124r, which are each connected to a strip conductor piece 125r.

Each of these strip conductor pieces 125r is connected by way of a special via to one of the strip conductors 26r, which are situated on the substrate front side 12 and to the right of the longitudinal axis LA. Details regarding this are described further below.

In a further exemplary embodiment, it is provided that contact sites are also provided on the rear side 14. These rear-side contact sites can be equal in size to the front-side contact sites 16 and also can be made virtually to coincide in position with them. In the case of the presence of contact sites on both sides 12, 14, an associated membrane connector should be used. Diverse contacting and circuitry variants are hereby possible.

The sensor resistors 128l, 128r shown in FIG. 5 have a design and arrangement that is identical—or at least essentially identical—to the design and arrangement of the sensor resistors 28l and 28r present on the substrate front side 12. Therefore, they cannot be seen in FIG. 1. It is hereby noted once again that, in FIG. 5, the illustration corresponds to a plan view of the substrate front side 12 with the view passing through the substrate 10 and the elements that are on the substrate front side 12 not being present.

FIG. 6 shows a symbolic enlargement of the region A marked in FIG. 1. What is hereby essentially involved is the illustration of a via between one of the front-side strip conductor pieces 25l and the associated strip conductor 126l, that is, the strip conductor 126l situated oppositely on the substrate rear side 14. FIG. 7 shows a symbolic cross-sectional illustration of this via.

It can be seen from FIGS. 6 and 7, in particular, that, on the front side 12 of the substrate 10, one of the left sensor resistors 28l and below it (in accordance with FIG. 7), on the substrate rear side 14, an associated sensor resistor 128l are arranged. Moreover, the front-side sensor resistor 28l is connected to the front-side contact point 24l and, in turn, the latter is connected to the front-side strip conductor piece 25l. To this end, it is noted that the contact point 24l and the strip conductor piece 25l represent a common electrical connecting element and are drawn separate here only for a simple description of the figures. The rear-side sensor resistor 128l is connected to the rear-side contact point 124l and, in turn, the latter is connected to the rear-side strip conductor 126l, which leads by way of a further via to the associated contact site 16 (see FIG. 1). Arranged between the front side 12 and the rear side 14 through a corresponding opening within the substrate 10 is a via conductor 200, which here consists of the same material as the front-side strip conductor piece 25l and the rear-side strip conductor 126l and electrically interconnects these two elements 25l, 126l. Accordingly, the two sensor resistors 28l and 128l are connected in series. Moreover, even though not illustrated for reasons of clarity, it ensues from the preceding explanations as well as from FIGS. 1 to 5 that
the apparently free end 29l of the sensor resistor 28l is electrically connected via the contact point 22l to the supply voltage conductor strip 20 and
the apparently free end 1291 of the sensor resistor 128l is electrically connected via the contact point 122l to the supply voltage conductor strip 120.

The other ones of the front-side left sensor resistors 28l are also connected in such a way to their associated rear-side sensor resistors 128l. The same also applies to the right sensor resistors 28r and 128r.

The sensor resistors 28, 128 are preferably produced by a screen printing method, in which, in this case, a carbon-based paste, structured with a thickness of approximately 5 to 20 μm, is applied to the two sides 12, 14 of the substrate 10. In the exemplary embodiment described, the sensor resistors 28, 128 hereby have a length of approximately 7 mm. Their width can be quite different depending on the application and lies in the range of approximately 100 μm to 800 μm in order to realize resistance values in the range of between 10 kΩ and 80 kΩ. The other elements present on the substrate sides 12, 14, such as, in particular, the strip conductors and the contact points, are preferably likewise produced by a screen printing method, in which silver paste with a thickness of approximately 5 to 15 μm is applied.

FIG. 8 shows a circuit diagram, in which a pair of left sensor resistors 28l, 128l and a pair of right sensor resistors 28r, 128r are taken into account The circuit diagram is divided into the following three blocks:
S: sensor region
K: contact region
A: evaluation unit

The sensor region S essentially corresponds to the part of the sensor strip 9 in which the sensor resistors 28l, 28r, 128l, 128r are arranged. It is designed in such a way that it is flexible and, in particular, can be bent, twisted, and/or stretched. The contact region K essentially corresponds to the part of the sensor strip 9 in which the supply voltage conductor strips 20, 120 as well as the strip conductors 26r, 126l are connected to the contact sites 16. In a preferred embodiment, the contact region K (see also FIG. 1), at least in the operating mode (normal operation), is substantially less flexible or deformable than the sensor region S. This can be achieved, for example, by way of the terminal of a membrane connector at the contact sites 16, by way of a lesser bonding of the region K to the object that is to be examined. and/or way of a corresponding design of the substrate 10 (such as, its thickness, material, etc.).

The evaluation unit A, which here is connected via the contact 300 of a membrane connector to the contact sites 16, contains a supply voltage source as well as conventional electronic elements, such as, for example, amplifiers, A/D converters, memory storage components, transmitting devices, display elements, and/or the like. This will be addressed in detail further below.

As ensues from the circuit diagram of FIG. 8, a supply voltage +u is applied to the front-side supply voltage conductor 20, the value +U of which can be, for example, 3 volts. The rear-side supply voltage conductor 120 is at ground. The sensor resistors 28r and 128r are connected in series between the supply voltage +u and ground, so that they form a voltage divider, with the strip conductor 26r making possible the center tap. The sensor resistors 28l and 128l also form a voltage divider of this kind, with the strip conductor 126l making possible the center tap. The two strip conductors 26r, 126l each lead to one of the contact sites 16. Via an associated membrane connector contact 300, the divided voltages, which are also referred to below as sensor signals sr and sl, are conveyed to amplifier stages 302a and 302b, respectively. The output signals thereof are fed to an evaluation stage 304, which, on account of the amplified sensor signals, generates output signals, which are conveyed via a signal conductor 308 to a display stage 306.

In FIG. 8, for reasons of clarity, the processing of a sensor signal sl, which originates from a pair of left resistors 28, 128l, as well as the processing of a sensor signal sr, which originates from a pair of right resistors 28r, 128r, are illustrated. It is obvious that the evaluation unit A can also receive and process the sensor signals of the other resistor pairs 28l, 128l and 28r, 128r. There exist various possibilities for this, such as, for example, the use here of a switching stage, which is not depicted, inside of or outside of the evaluation unit A. Such a switching stage could receive a plurality of or all of the sensor signals sl, sr and convey them further in accordance with the principle of a time multiplexer under time control via the amplification stages 302a, 302b to the evaluation stage 304. It is also conceivable that, for each of the sensor signals sl, sr, a separate amplifier stage 302a and 302b, respectively, is provided and the amplified sensor signals are conveyed to the evaluation stage 304 by means of an associated switching stage. It is also conceivable that each of the sensor signals sl, sr (that is, in this exemplary embodiment, 5×sl+5×sr) is fed to a separate amplifier stage 302a, b and the evaluation stage 304 has a total of 10 inputs for the amplified sensor signals. Further possible are also mixed forms of the mentioned alternatives.

As already mentioned, the individual ones of the resistor pairs 28l, 128l and 28r, 128r form series circuits and accordingly voltage dividers. Accordingly, the following holds for the value of the sensor signals sl, sr:

SL=+U*R128l/(R28l+R128l)

SR=+U*R128r/(R28r+R128r)

with
SL: voltage value of the sensor signals sl with respect to ground
SR: voltage value of the sensor signals sr with respect to ground
+U: value of the voltage +u with respect to ground
R128l: value of the sensor resistor 128l
R28l: value of the sensor resistor 28l
R128r: value of the resistor 128r
R28r: value of the resistor 28r.

Ideally—that is, in particular, without taking into consideration manufacturing tolerances—the sensor resistors 28l, 28r, 128l, 128r are designed in such a way that their values are then equal in size when the sensor strip 9 is situated without mechanical strains in a plane (such as, for example, in the x-y plane of FIG. 1). Such a state is also referred to here as the resting state. The following then holds:

R28l=R128l=R28r=R128r.

In such a case, the following then holds as well:

SL=½*+U and

SR=½*+U.

When the sensor region S arches backward out of the x-y plane (in the −z direction), the front-side resistors 28l, 28r are stretched and the rear-side resistors 128l, 128r are compressed. The effect on the individual resistors is hereby dependent on their angle of inclination α as well as on the location and direction of the arching. This is explained briefly with the help of FIG. 1, with it being assumed that the sensor strip 9 is arched uniformly downward perpendicular to the longitudinal axis LA (that is, it does not have any kink, but rather has the shape of a semicircular arch) and the angles of inclination α have a value of between zero and 90 degrees. The effect on the individual resistors is then all the stronger, the smaller is the angle of inclination α. When, in contrast, there is a uniform arching parallel to the longitudinal axis LA, the effect on the individual resistors is all the stronger, the larger is the angle of inclination α.

In the case of a nonuniform arching of the sensor region S (the extreme case is a kink), the effect on an individual one of the resistors 28, 128—in addition to the effect on its angle of inclination α—is also very dependent on whether and how it is situated in the region of the arching.

When the sensor region S arches forward out of the x-y plane (in the z direction), the front-side sensor resistors 28l, 28r are compressed and the rear-side sensor resistors 128l, 128r are stretched. When the sensor region S is brought into a wave shape, it is possible for individual ones of the front-side sensor resistors 28l, 28r to be compressed and for others to be stretched.

When a twisting (torsion) of the sensor strip 9 or of its sensor region S occurs, changes in resistance also arise, which are dependent on the position of the individual sensor resistor 28, 128 as well as on its angle of inclination α. When, for example, the lower part of the sensor strip 9—that is, the region in the direction of the contact sites 16—remains in the plane of the drawing and its upper part is twisted in the clockwise direction corresponding to the torsion arrow TP (FIG. 1), the left front-side resistors 28l are stretched and the left rear-side resistors 128l are compressed. On the right side, in contrast, the front-side resistors 28r are compressed and the rear-side resistors 128r are stretched. The extent of such a stretching or compression is hereby dependent on the angle of inclination α as well as also on the characteristics of the substrate. It has hereby been found that, in the case of a relatively thin substrate, corresponding essentially to a film, the resistance values change the most when the angle of inclination α has a value of approximately 45 degrees. Of course, such a change is further dependent on how strong the torsion is at the location of the respective resistor. Accordingly, the following can be stated in general: It is possible by way of the angle of inclination α to adjust the ratio of the sensitivity of the resistor pairs with respect to the torsion to the sensitivity of the resistor pairs with respect to the bending transverse to the longitudinal axis LA. By way of the combination of at least two differently angled resistor pairs, it is accordingly possible to measure a bending direction that does not extend transverse to the longitudinal axis LA or an overlapping torsion that extends transverse to the bending extending transverse to the longitudinal axis LA.

When the sensor region S is stretched, this likewise has consequences for the individual sensor resistors. These consequences are dependent on the position and the orientation of the individual resistor as well as on the kind and direction of the stretching. Converse effects arise when the sensor region S is compressed. In the preferred exemplary embodiment, resistors that lie opposite one another, such as the resistor pairs 28l, 128l or 28r, 128r, are similar in design. This means that they have essentially the same geometry and the same material properties. The result hereof is that a stretching of the sensor region S has the same consequences for the oppositely lying resistors and therefore the corresponding values SL, SR ideally do not change. However, if the respectively oppositely lying resistors differ, then, in the case of a stretching, a change in the values SL, SR is possible.

On the basis of the described examples, it becomes clear that the individual resistance values R28l, R128l, R28r, R128r are dependent on the mechanical influencing of the sensor strip 9 or of its sensor region S. Conversely, it ensues that, in the event that these resistance values are changed—and, accordingly, the associated sensor signal value SL, SR is changed—a corresponding mechanical influencing of the sensor strip 9 exists. To this end, in the evaluation stage 304, the amplified sensor signals sl, sr are evaluated as a function of the position and orientation of the associated sensor resistors 28l, 128l, 28r, 128r on the basis of a suitable algorithm. To this end, the evaluation stage 304 has conventional elements known to the person skilled in the art, such as a microprocessor, an analog-digital (A/D) converter, memory storage components, etc.

As described above, the sensor signals sl, sr are each the result of the center tap in the case of a voltage divider having two resistors. It is also possible that, instead of absolute voltages, a differential voltage is measured and processed in each case. Such a differential voltage can be produced, for example, by means of a Wheatstone bridge, in which the reference voltage required for such a differential voltage is usually generated by a suitable second voltage divider. When the value of the first voltage—generated by one of the voltage dividers 28, 128—is now identical in the resting state to the value of the reference voltage, the difference is zero. When, in the subsequent measurement operation, the sensor region S experiences a mechanical movement in such a way that one of the two resistors 28, 128 changes, there is also a change in the value of the tapped voltage. However, this change in voltage can be relative—that is, in relation to the absolute voltage value—quite small. A change in voltage of this kind can be evaluated markedly better when not the tapped voltage as such of the evaluation stage 304 is processed, but rather the differential voltage produced by means of the reference voltage is processed, since the relative change thereof is substantially larger than that of the tapped voltage.

In an exemplary embodiment in accordance with the invention, the evaluation stage A is designed in such a way that a reference voltage is generated, the value of which corresponds exactly or at least essentially to the voltage value than results from the respective voltage divider 28, 128, such as preferably ½*+U (see above). Within the amplification stages 302a, 302b—but prior to the actual amplification—the difference between the sensor signal sl and the reference voltage as well as the difference between the sensor signal sr and the reference voltage are generated. In order to avoid negative voltages, a voltage is added after the amplification in each case, the value of which is preferably ½*+U (1.5 V). The value of this voltage is again subtracted during the subsequent digitalization, so that also negative digital sensor values can result and can be used already through their sign to draw conclusions about the bending direction.

The result of the evaluation of the sensor signals sl, sr is output by using output signals sa. This result can be designed in various ways. Preferably, it contains the requisite information in order for the display stage 306, which is equipped with an associated display, to be able to reproduce the sensor strip 9 or, more precisely, the sensor region S graphically. It is hereby also possible for such a reproduction to assume a dynamic time course, which is dependent on the time point of the measurement. For this purpose, the evaluation stage 306 can have suitable memory storage components (not depicted separately) in order to be better able to record and later retrieve such a dynamic reproduction.

It is also conceivable that the output signal sa and/or the display stage 306 are or is designed in such a way that, in addition to or instead of a graphical display, acoustical and/or haptic (vibrations or the like) alert signals are output when the sensor region S is moved in such a way—for example, arched, twisted, and/or stretched—that predetermined threshold values are exceeded. The criteria for such threshold values can be very different. It can hereby also be taken into account when predetermined geometric values of the course of the speed thereof and/or the acceleration thereof are not attained or are exceeded for a certain time.

As mentioned, the display stage 306 can be designed in very diverse ways. It is also conceivable for it to be arranged to be outside of the evaluation unit A and to be designed, for example, as a PC, a tablet computer, a smartphone, or the like. It is hereby also possible for the algorithm for evaluation of the sensor signals sl, sr to be run in part or in full on such a device.

The signal conductor 308 can be realized as a cable and/or in a cable-free manner. This means that the evaluation unit A has, as needed, a transmission stage. The latter can be designed in such a way that it can transmit high-frequency signals (for example, Bluetooth, WLAN, etc.), optical signals, acoustical signals, and/or the like. This is especially advantageous in the case when the display stage 306 is realized as a smartphone or a tablet computer.

The sensor strip according to the invention can be used in diverse ways, such as, for example, in
the field of orthopedics
medical diagnostics
smartwatches and other wearables
life science

A preferred application in the field of the orthopedics relates to the measurement of the spinal column of a human being. To this end, the sensor strip 9 is placed by suitable means, such as patches, adhesive, or the like, on the back of the human being in question. It is also conceivable that, for this purpose, a kind of clothing piece is used, such as a T-shirt, a body shirt, a vest, or the like, in which and/or on which the sensor strip 9 is arranged or integrated. By way of individual measurements or, better still, by way of ongoing measurements within a certain period of time, it is possible to determine how the back moves and, if need be, how it is subjected to loads. For this purpose, a special embodiment of the sensor strip according to the invention has proven to be especially expedient and will be described below as the sensor strip 900 on the basis of FIGS. 9 to 11. FIG. 9 hereby shows a plan view of the entire sensor strip 900 and FIGS. 10 and 11 show sections B and C thereof, which, in FIG. 9, are marked. The sensor strip 900 also consists of a substrate (not shown here) with a front side and a rear side. On account of the plan view, essentially the elements that are situated on the substrate front side are depicted and the elements that are situated on the substrate rear side and are covered up by the substrate front side elements are not visible here.

Elements that are identical or similar to those of the previously described sensor strip 9 are given the same reference signs and they will be addressed only insofar as they seem to be required in order to understand the present invention. In addition, reference is made to the above embodiments in regard to the sensor strip 9.

The sensor strip 900 shown in FIG. 9 has a sensor region S that is approximately 60 cm long. Hereby present on the substrate front side 12, along the longitudinal axis LA, is a front-side strip conductor 18, to the left 28 of which a piece of sensor resistor 28l and to the right of which 28 a piece of sensor resistor 28r are electrically connected. Here, they are also oblong in design and have a nearly rectangular shape and all here have the same angle of inclination α, the value of which is approximately 30 degrees. The left sensor resistors 28l are each connected to one of the left strip conductors 26l and the right sensor resistors 28r are each connected to one of the right strip conductors 26r.

The sensor resistors 128l, 128r arranged on the substrate rear side 14 are situated precisely (or essentially) below the front-side sensor resistors 28l, 28r and are therefore not visible here. Analogously to the front-side sensor resistors 28l, 28r, they are connected, on the one hand, to the rear-side supply voltage conductor strip 120, only a small sectional cutout of which can be seen here above the region 52 (see FIG. 11), and, on the other hand, to the rear-side strip conductors 126l and 126r, which extend on the substrate rear side 14 opposite to or below the left strip conductors 26l or 26r.

This means that, in the case of the sensor strip 900, a separate strip conductor 26, 126 is provided for each of the sensor resistors 28, 128. Thus, this is different from the case for the sensor strip 9, in which, for each pair of resistors 28, 128, initially one of the vias 200 is provided and afterwards only one of the strip conductors 26r, 126l is provided. The required vias 200 for the realization of voltage dividers from two resistors 28, 128, lying one above the other, are all situated in the case of the sensor strip 900 in a region 902 (see FIG. 11), which is situated in the region of the contact sites 16 and thus inside of the contact region K and accordingly lies outside of the sensor region S (see FIG. 9). Through the arrangement of the vias 200 outside of the sensor region S, which, in normal measurement operation, is usually bent, twisted, and/or stretched, a very robust sensor strip 900 is achieved.

The sensor strip illustrated in FIGS. 9 to 11 can be shortened in that a desired length is cut off the upper part. In this way, although the number of resistor pairs decreases, the sensor strip length can be adapted to the object that is to be examined.

In a further exemplary embodiment, which is not illustrated here by figures, it is provided that
the mentioned vias 200 are arranged near to the individual resistor pairs (voltage divider) 28, 128 (similarly to the case of the sensor strip 9), so that, per resistor pair, only one of the strip conductors 26, 126 is required and, furthermore, that
a first number of these resistor pairs 28, 128 are connected via front-side strip conductors 26 and the remaining resistor pairs via rear-side strip conductors 126 to the contact sites 16.

This affords the advantage that such a sensor strip can be designed to be narrower than the sensor strip 900. However, it needs hereby to be taken into account that, in the case of one-sided contact sites 16 (membrane connectors), additional vias are required.

Shown in FIG. 12, which is similar in part to FIG. 7, is a further exemplary embodiment. The essential difference from the previously described embodiments consists in the fact that here the front-side strip conductor 251 is connected to the rear-side strip conductor 126l via a first contact element 252 and a second contact element 254 as well as a connecting conductor 256 between the contact elements 252 and 254. For a simple and operationally reliable contacting, the front-side strip conductor 251 extends all the way to a first contact site 251, which is situated in the edge region of the front side 12 of the substrate 10. Correspondingly, the rear-side strip conductor 1261 extends all the way to a second contact site 253, which is situated in the edge region of the rear side 14 of the substrate 10. Preferably, the two contact sites 251, 253 are arranged in such a way that they lie opposite to each other. In normal operation (as also shown here), the first contact element 252 is in electrical contact with the first contact site 251 and accordingly with the strip conductor 251 and the second contact element 254 is in electrical contact with the second contact site 253 and accordingly with the strip conductor 1261. Moreover, the connecting conductor 256 is connected to the evaluation unit A, at which a corresponding sensor signal sl (see also FIG. 8) is emitted.

It is obvious that the electrical connection shown here between the front-side strip conductor 251 and the rear-side strip conductor 126l by means of the electrical contact elements 252, 254 is only shown by way of example. By way of contacts of this kind and/or similar contacts, it is also possible to connect further ones or even all of the front-side strip conductors to the associated rear-side strip conductors, so that the number of vias 200 can be reduced correspondingly or they can be dispensed with entirely.

The described exemplary embodiments are only given by way of example and represent preferred realizations of the present invention. It is obvious that specific features, which were described above in connection with individual exemplary embodiments, can also be used in the case of other exemplary embodiments and insofar there are no obvious limitations imposed against them.

Moreover, diverse variations and alternatives of the described embodiments are possible such as, for example:
The supply voltage +u can be a direct current voltage and/or an alternating current voltage.
The elements that are here referred to as the resistors 28, 128 can also be used as capacitors. To this end, these elements are to be correspondingly connected to the evaluation unit and an appropriate alternating current voltage is to be applied.

### LIST OF REFERENCE CHARACTERS

9 first sensor strip
10 substrate
12 front side of 10
14 rear side of 10
16 contact sites
18 strip conductors on 12
20 front-side supply voltage connector strip
22l, 22r left and right contact points on 12
24l, 24r left and right contact points on 12
25l strip conductor piece on 12
26l, 26r left and right strip conductors on 12
28l, 28r left and right sensor resistors on 12
29l an end of 281
50 strip conductor on 12
52 region of a via from 50 to 120
120 rear-side supply voltage strip conductor
122l, 122r left and right contact points on 14
124l, 124r left and right contact points on 14
125r strip conductor piece on 14
126l left strip conductors on 14
128l, 128r left and right sensor resistors on 14
129l an end of 128l
200 via conductor between 251 and 126l
251 first contact site
252 electrical contact element to 251
253 second contact site
254 electrical contact element to 253
256 connecting conductor between 252 and 254
300 circuit board connector contacts
302a, b amplification stages
304 evaluation stage
306 display stage
308 signal conductor
900 second sensor strip
902 via region
LA longitudinal axis of 9 or 900
TP torsion arrow
S sensor region
K contact region
A evaluation unit
lal, lar longitudinal axis of a left and right sensor resistor
sl, sr left and right sensor signal
sa output signal
α angle of inclination

In the claims:

1. A sensor strip for measuring geometric shapes, wherein
a substrate
at least one first pair of two electrical resistors are provided, the first of which being arranged on the front side of the substrate and the other resistor being arranged on the rear side of the substrate in such a way that it lies essentially opposite to the first resistor
the first terminal of the first resistor is electrically connected via a first supply voltage conductor to the first pole of a supply voltage and the first terminal of the second resistor is electrically connected via a second supply voltage conductor to the second pole (ground) of the supply voltage
the second terminals of the two resistors are electrically interconnected, so that they form a voltage divider,
is hereby characterized in that,
for the electrical connection between the second terminals of the two resistors, at least one via is provided, which electrically connects a strip conductor to the second terminal of the first resistor on the front side to a strip conductor to the second terminal of the second resistor on the rear side, and/or in that the two strip conductors lead to respective contact sites, which are designed and arranged in such a way that they can be electrically interconnected via contact elements.

2. The sensor strip according to claim 1, further characterized in that
at least one second pair of two electrical resistors is provided, the first of which being arranged on the front side of the substrate and the other resistor being arranged on the
rear side of the substrate in such a way that it lies essentially opposite to the first resistor
1. the first terminal of the first resistor is electrically connected via the first supply voltage conductor to the first pole of the supply voltage and the first terminal of the second resistor is electrically connected via the second supply voltage conductor to the second pole of the supply voltage
2. the second terminals of the two resistors are electrically interconnected, so that they form a voltage divider
3. at least one of the first resistor pairs and at least one of the second resistor pairs are arranged adjacent to each other.

3. The sensor strip according to claim 1, further characterized in that the first resistor pairs and/or the second resistor pairs have an angle of inclination with respect to normals, the value of which lies between zero and 90 degrees.

4. The sensor strip according to claim 1, further characterized in that the number of first resistor pairs is equal to the number of second resistor pairs and they are arranged in mirror symmetry with respect to each other in relation to the normals.

5. The sensor strip according to claim 1, further characterized in that the first supply voltage conductor is arranged on the front side of the substrate and extends between the first resistor pairs and the second resistor pairs, preferably along the normals.

6. The sensor strip according to claim 1, further characterized in that the second supply voltage conductor is arranged on the rear side of the substrate and extends between the first resistor pairs (28l, 128l) and the second resistor pairs (28r, 128r), preferably along the normals.

7. The sensor strip according to claim 1, further characterized in that at least individual ones of the resistors are applied to the substrate by a printing method, such as, for example, screen printing, and preferably a high-ohmic paste is used for this purpose.

8. The sensor strip according to claim 1, further characterized in that at least individual ones of the connecting conductors required for the terminals of the resistors and/or at least one of the vias are applied to the substrate by a printing method, such as, for example, screen printing, and preferably a low-ohmic paste is used for this purpose.

9. The sensor strip according to claim 1, further characterized in that it has a first region, which, in normal operation, is deformable and in which the mentioned resistors are arranged, and it has a second region, which, in comparison with first region, is less deformable in normal operation and in which at least individual ones of the vias are arranged.

10. A device for measuring geometric shapes, characterized in that a sensor strip according to one of the above claims is used, and in that the resistors are connected via suitable electrical connections to an evaluation unit, which delivers the supply voltage and wich receives voltages produced by the voltage dividers as sensor signals and generates an output signal, which is a measure for changes in resistance within the individual resistor pairs.

11. The device according to claim 10, further characterized in that the evaluation unit generates a reference voltage, the value of which essentially corresponds to the value that the sensor signals have in a resting state of the sensor strip, and forms a difference between the value of this reference voltage and the value of the measured sensor signals.

12. The device according to claim 10, further characterized in that the sensor signals of at least individual ones of the resistor pairs are evaluated in succession, so that, taking into consideration their position inside of the sensor strip, an output signal is generated, the value of which is a measure for the geometric shape of the sensor strip.

13. The device according to claim 10, further characterized in that, successively in time, sensor signals of at least individual ones of the resistor pairs are evaluated, so that, taking into consideration their position inside of the sensor strip, an output signal is generated, the value of which is a measure for the course of movement of the resistor pairs in question.

14. The device according to claim 10, further characterized in that it has a transmission unit, which emits a transmission signal as a function of the output signal.

15. The device according to claim 10, further characterized in that it has at least one pair of contact elements, with one of these contact elements being designed and arranged in such a way that, in normal operation, it is in electrical contact with one of the front-side strip conductors and the other one of these contact elements being designed and arranged in such a way that, in normal operation, it is in electrical contact with one of the rear-side strip conductors, with the two contact elements being electrically interconnected.
